(19) United States
(12) Patent Application Publication
(10) Pub. No.: US 2016/0152949 A1
(43) Pub. Date: Jun. 2, 2016

(54) METHOD AND CULTURE MEDIUM FOR IN VITRO CULTURING OF STEM CELLS

(71) Applicant: ERASMUS UNIVERSITY MEDICAL CENTER ROTTERDAM, Rotterdam (NL)

(72) Inventors: Derk Ten Berge, Rotterdam (NL); Johannes Jacobus Comelissen, Rotterdam (NL); Nesrin Tuysuz, Rotterdam (NL); Gerrit Storm, Rotterdam (NL)

(21) Appl. No.: 14/905,594

(22) PCT Filed: Jul. 15, 2014
(86) PCT No.: PCT/NL2014/050482

§ 371 Date: Jan. 15, 2016

(30) Foreign Application Priority Data
Jul. 15, 2013 (NL) 2011170

(51) Int. Cl. C12N 5/071; A61K 35/37

(52) U.S. Cl. CPC C12N 5/068; A61K 35/37; C12N 2501/415; C12N 2500/36

(57) ABSTRACT

The present invention relates to a serum-free culture medium for the culture of adult stem cells comprising a Wnt protein and a lipid. The invention further comprises a method for culturing adult stem cells using such a method and methods of stem cell therapy wherein the stem cells are cultures in said medium.

### FIELD OF THE INVENTION

The invention relates to the fields of tissue culture, more importantly the culture of adult, tissue specific stem cells and use of stem cells for therapy.

### BACKGROUND OF THE INVENTION

Adult stem cells are useful for therapeutic treatments of the tissues from which they have been generated. However, culturing of adult stem cells often is very challenging. One of the difficulties in this respect is that their environment can often be a cause of differentiation. This phenotypic instability represents a major challenge for maintaining adult stem cell cultures in vitro.

The environmental factor in tissue engineering is mainly formed by the medium and in the case of stem cell culture this medium normally would contain serum of mammalian origin. Said serum is—by definition—a source of unknown factors, like serum proteins and other influencing compounds that have been excreted into and/or transported by the blood. It is very important that the amount of unknown components is minimalized in therapeutic applications of stem cells, because introduction of biological material into a patient's body should be as controlled as possible. For embryonic stem cells serum-free media have been developed in the mean time (see e.g. Vallier, L., 2011 Meth. Molec. Biol., 690:57-66). For adult human stem cell lines a serum-free culture medium has not yet been suggested.

Further, stem cell media should be able to provide all the nutrients and other compounds that are essential for the multiplication of the stem cells, but they may not contain compounds that would be detrimental to the growth or multiplication of the stem cells or that would give lead to an undefined further differentiation of the stem cells into specific cells. Over the past 15 years, the Wnt signaling pathway has been shown to regulate self-renewal and cell fate choices of both embryonic stem cells and a variety of adult tissue stem cells, such as those from the gastrointestinal system, skin and hair, and nervous system (Clevers, H. and Nusse, R., 2012, Cell 149:1192-1205). These data indicate that Wnt signals would be beneficial for the self-renewal of stem cells in culture and may offer a way for the in vitro manipulation of stem cells prior to their reintroduction into patients. Culture systems for several human and mouse adult stem cells have recently been defined Huch, M. et al., 2013, Nature 494:247-250). These systems rely on an agonist of the Wnt pathway, R-Spondin1, that acts by binding the Lgr5 receptor and thereby enhances the activation of the Wnt signaling pathway by Wnt proteins. For colon, gastric, liver and for human stem cells, endogenous Wnt signals are insufficient and exogenous Wnt3a is required. It was noticed however that purified Wnt3a protein proved less efficient at maintaining gastric organoids than did Wnt3a-conditioned medium containing serum (Barker, N. et al., 2010, Cell Stem Cell 6:25-36). However, as indicated above, in clinical applications the presence of serum is undesired.

The Wnt proteins are a group of secreted lipid-modified (palmitoylation) signaling proteins of 350-400 amino acids in length. Following the signal sequence, they carry a conserved pattern of 20-24 cysteine residues, on which palmitoylation occurs on a cysteine residue. These proteins activate various pathways in the cell that can be categorized into the canonical and noncanonical Wnt pathways. Through these signaling pathways, Wnt proteins play a variety of important roles in embryonic development, cell differentiation, and cell polarity generation. The human Wnt3a gene is a member of the WNT gene family. It encodes a protein showing 96% amino acid identity to mouse Wnt3A protein, and 84% to human WNT3 protein, another WNT gene product. The Wnt3a gene is clustered with WNT14 gene, another family member, in chromosome 1q42 region.

Wnt proteins are soluble signaling molecules that require attachment of a lipid moiety in order to gain activity, and are for this reason hydrophobic (Willert, K. et al., 2003, Nature 423:448-452). They are therefore purified in the presence of detergents that maintain their solubility. However, upon dilution in cell culture medium the detergent concentration is insufficient to maintain Wnt solubility which then rapidly loses activity, in particular in the absence of serum Fuerer, C. et al., 2010, Dev. Dyn. 239:184-190).

Currently, human adult stem cells can not be efficiently derived and/or maintained in the absence of serum because this results in insufficient Wnt activity in the culture. High Wnt activity in stem cell cultures may be maintained in several ways:

frequent replenishment of media, which adds dramatically to costs and also interrupts the culturing, which is generally undesirable;

adding serum, which is an undefined product, interferes with clinical applications and induces differentiation of many stem cells;

using Wnt conditioned medium (see experimental part for definition) in stead of purified Wnt. However, Wnt in conditioned medium has the same disadvantages as serum;

possibly adding Wnt-stabilizing compounds such as glucosaminoglycans. Next to being extremely expensive no beneficial effects of addition of glucosaminoglycans have yet been demonstrated.

Thus, there is need to develop a more defined medium for culturing adult human stem cells, in which the proliferation of the cells is enhanced and the differentiation of cells is inhibited. Such a medium would preferentially comprise one or more Wnt proteins that would remain active for a long time and this medium would need to be free of serum or other undefined components. The availability of such a serum-free adult human stem cell culture medium would also enable further use of such adult stem cells.

### SUMMARY OF THE INVENTION

The invention is directed to a method for in vitro culturing of stem cells, wherein the cells are held in a serum-free culture medium comprising a Wnt protein and a lipid, wherein said lipid is available in a concentration of at least 0.1 mM. Preferably in said method the lipid is in the form of a liposome or of a micelle, more preferably, the lipid and the Wnt protein are associated in a complex. Preferably such a liposome is composed of dimyristoyl-sn-Glycero-3-Phosphocholine (DMPC), 1,2-dimyristoyl-sn-glycero-3-phospho-(1′-rac-glycerol)) (DMPG) and cholesterol, preferably in a DMPC:DMPG:cholesterol ration of 10:1:10.

In a further preferred embodiment the Wnt protein is selected from the group of human Wnt protein, preferably wherein the protein is Wnt3a. In a further preferred embodiment the stem cells are adult stem cells, preferably intestinal stem cells, more preferably stem cells obtained from duodenum and/or ileum. Further preferred is a method according to the invention wherein the stem cell culture is an organoid culture. The invention also comprises a serum-free culture medium for the culture of stem cells comprising a Wnt protein and a lipid wherein said lipid is available in a concentration of at least 0.1 mM, preferably wherein said lipid is in the form of a liposome or micelle.

Further part of the invention is a method for stem cell therapy, comprising the steps of:
a. isolation of stem cells from a subject;
b. optionally treating said stem cells, wherein the treatment may be chosen from differentiating, dedifferentiating, redifferentiation, reprogramming, introducing of a mutation, genetic modification;
c. culturing said stem cells in a serum-free culture medium according to the invention;
d. introducing said cultured stem cells in a subject in need thereof.
   Also comprised in the invention is a method for autologous adult stem cell therapy comprising the steps of:
a. isolation of adult stem cells from a subject;
b. culturing said adult stem cells in a serum-free culture medium according to the invention;
c. re-introduction of said cultured adult stem cells in said subject.
   Further, the invention comprises a method for autologous adult stem cell therapy comprising the steps of:
a) isolation of adult stem cells from a subject;
b) genetic modification of said adult stem cells;
c) culturing said genetically modified adult stem cells in a serum-free culture medium according to the invention;
d) re-introduction of said cultured adult stem cells in said subject.
   Also, the invention includes a method for adult stem cell therapy comprising the steps of:
a. isolation of adult stem cells from an organ of a subject;
b. genetic modification of said adult stem cells;
c. culturing said genetically modified adult stem cells in a serum-free culture medium according to the invention;
d. re-introduction of said cultured adult stem cells in said subject, wherein said adult stem cells are genetically modified in such a way that they are producing a therapeutic compound for treatment of a disease wherein said disease is not or only partly related to the organ or the organ system from which the adult stem cells are derived in step a.
   The invention further comprises a method for adult stem cell therapy comprising the steps of:
a. isolation of adult stem cells from an organ of a subject;
b. genetic modification of said adult stem cells;
c. re-introduction of said cultured adult stem cells in said subject,

wherein said adult stem cells are genetically modified in such a way that they are producing a therapeutic compound for treatment of a disease wherein said disease is not or only partly related to the organ or the organ system from which the adult stem cells are derived in step a.

The invention further comprises a serum-free medium comprising a Wnt protein and a lipid for a for adult stem cell therapy, wherein the adult stem cell therapy comprises:
a. isolation of adult stem cells from a subject;
b. culturing said adult stem cells in a serum-free culture medium according to the invention;
c. re-introduction of said cultured adult stem cells in said subject.

Also comprised in the invention is a serum-free medium comprising a Wnt protein and a lipid for a for adult stem cell therapy, wherein the adult stem cell therapy comprises:
a. isolation of adult stem cells from a subject;
b. genetic modification of said adult stem cells;
c. culturing said adult stem cells in a serum-free culture medium according to the invention;
d. re-introduction of said cultured adult stem cells in said subject.

### BRIEF DESCRIPTION OF THE DRAWINGS

FIG. 1: Wnt3a conditioned medium but not purified Wnt3a protein supports the derivation of intestinal stem cell organoids. Derivation of human duodenum and ileum organoid cultures in the presence of Wnt3a conditioned medium, purified Wnt3a, or purified Wnt3a and serum. Pictures are from organoid cultures taken after the first passage since derivation.

FIG. 2: Wnt3a protein activity is rapidly lost in serumfree cell culture medium. Wnt3a-conditioned medium (50%) or purified Wnt3a (250 ng/ml) is incubated in DMEM, in the presence or absence of 10% fetal calf serum as indicated, at 37° C. for the indicated amounts of time. The remaining Wnt3a activity is then determined using the LSL assay. CM: conditioned medium.

FIG. 3: Short half-life and detergent-associated toxicity limits the use of purified Wnt3a to support stem cell self-renewal. A) Serum free ES cell self-renewal assay comparing the effect on self-renewal of addition of 250 ng/ml purified Wnt3a daily or after passaging every 3 days. Self-renewal is much reduced when Wnt3a is added after every passage, indicating that the rapid loss of Wnt3a activity in serum free culture limits self-renewal and necessitates frequent replenishment with fresh Wnt3a. B) Purified Wnt3a protein seems to inhibit ES cell self-renewal when present above a concentration of 500 ng/ml. However, this appears to result from detergent-associated toxicity as lower concentrations of Wnt3a with equivalent amounts of the detergent CHAPS also inhibit self-renewal.

FIG. 4: Wnt3a protein efficiently associates with liposomes, which enhances its stability. A) Liposomes of various compositions associated with Wnt3a protein were spun down by ultracentrifugation, and pelleted liposomes and supernatants analyzed by Western blot for the presence of Wnt3a. B) Quantification of the Western blot shown in A). C) Residual levels of CHAPS in Wnt3a liposomes following dialysis. D) Wnt3a activity half-life assays of different compositions of Wnt liposomes.

FIG. 5: ESC self-renewal assay comparing purified Wnt3a and Wnt3a liposomes. Wnt3a liposomes perform similar to purified Wnt3a protein when media were refreshed daily. However, when media were only refreshed following passaging every 3 days, Wnt3a liposomes supported a higher level of ES cell self-renewal than purified Wnt3a. Final concentration of Wnt3a protein is 250 ng/ml in all conditions.

FIG. 6: A) Epifluorescence images of R1-7xTcf-eGFP cells cultured for 3 days after the addition of 250 ng/ml purified Wnt3a, Wnt3a liposomes (250 ng/ml final concentration of Wnt3a), or vehicle liposomes. While reporter activity has declined when using purified Wnt3a protein, Wnt3a liposomes maintain strong reporter activity. B) R1-7xTcf-eGFP cells were cultured for the indicated amount of time in the presence of 250 ng/ml purified Wnt3a protein, Wnt3a liposomes (250 ng/ml final concentration of Wnt3a), or vehicle liposomes, and analyzed by flow cytometry for eGFP expression. When reagents were refreshed daily, both purified Wnt3a and Wnt3a liposomes maintained strong reporter expression. However, when reagents were not refreshed, reporter activity started to decline 2 days after purified Wnt3a addition, while Wnt3a liposomes maintained strong reporter activity even after 4 days.

FIG. 7: Wnt3a liposomes greatly enhance the establishment of intestinal stem cell organoids and suppress spontaneous differentiation. Derivation of human duodenum and ileum organoid cultures in the presence of Wnt3a conditioned medium, purified Wnt3a and serum, or Wnt3a liposomes. Many more organoids are obtained when Wnt3a liposomes are used. Moreover, signs of differentiation, such as a rough appearance of the organoids, signs of budding and irregular shape, are also greatly reduced in the serum free conditions containing Wnt3a liposomes. Pictures are from organoid cultures taken after the first passage since derivation.

FIG. 8: Liposomes stabilize Wnt3a protein activity when present in a wide range of concentrations. Different amounts of DMPC:DMPG:Cholesterol 10:1:10 liposomes were added to serum free medium, and purified Wnt3a protein was added separately at a final concentration of 500 ng/ml. Half-life activity assays showed that the liposomes stabilize Wnt3a protein activity through the entire concentration range tested.

### DETAILED DESCRIPTION OF THE INVENTION

“Adult stem cells” or “organ stem cells” as used herein are stem cells that are found throughout the body after development and are able to multiply, maintain tissue homeostasis, and regenerate damaged tissues. They are capable of prolonged self-replication and can differentiate all or most of the cell types of the organ from which they have been obtained. To indicate this feature they are also known as “multipotent stem cells”. Some adult stem cells are unipotent, e.g. spermatogonial stem cells. In culture, they are sometimes able to form so called “organoids” that mimic the tissue organisation of the tissue of origin, and which contain stem cells and differentiated offspring. Some of the differentiated offspring produce growth factors that promote self-renewal and expansion of the stem cells, allowing their expansion and propagation. Some stem cells may not form organoids in certain culture conditions but can nevertheless expand in favorable culture conditions. Because these stem cells or organoids containing stem cells can be expanded indefinitely from single stem cells, this technology is able to present a safe avenue of gene therapy. Especially since the offspring of individual stem cells can be analysed at the clonal level, which gives the opportunity that stem cells may be genetically altered and cultured and that the offspring may be selected for those stem cells that do not contain harmful mutations, which in turn can be expanded for subsequent transplantation.

The Wnt signaling pathway regulates a variety of cellular processes during the development of vertebrates and invertebrates, including cell proliferation and differentiation, cell fate, and organogenesis. In addition, the pathway controls tissue homeostasis and regeneration in response to damage in zebra fish, Xenopus, planarians, and in mammals including adult humans.

Wnt signaling is initiated by interaction of Wnt proteins with a variety of receptors, including members of the Frizzled (Fz) family of transmembrane receptors and members of the low-density-lipoprotein receptor-related protein (LRP) family (e.g., LRP5/LRP6). The extracellular Wnt signal stimulates intracellular signal transduction cascades including the canonical pathway, which regulates gene expression in the nucleus (see Logan C Y and Nusse, R. Annu. Rev. Cell Dev. Biol., 20:781-810, 2004) and several non-canonical pathways (reviewed by Kohn, A D and Moon, R T, Cell Calcium, 38: 439-446, 2005). Briefly, Wnt signaling via the canonical pathway leads to stabilization and nuclear localization of beta-catenin, which assembles with members of the T-cell factor/lymphoid enhancer factor (TCF/LEF) family of transcription factors to form complexes that generally activate transcription. In the absence of Wnt signaling, beta-catenin is instead targeted for degradation by the beta-catenin destruction complex, and TCF/LEFs form complexes that generally repress transcription. In the absence of Wnt signaling, kinases such as glycogen synthase kinase-3 (GSK3) and casein kinase 1 (CK1) phosphorylate beta-catenin, which as a consequence is ubiquinated and targeted for destruction by the proteasome. Activation of the Wnt pathway thus results in diminished phosphorylation of beta-catenin, thereby leading to its stabilization. Several endogenous proteins have been identified as inhibitors of Wnt signaling, including Dickkopf (Dkk), breakpoint cluster region protein (Bcr), proteins comprising a WIF (Wnt inhibitory factor) domain etc.

The term “Wnt” or “Wnt protein” refers to a polypeptide having a naturally occurring amino acid sequence of a Wnt protein or a fragment, variant, or derivative thereof that at least in part retains the ability of the naturally occurring protein to bind to Wnt receptor(s) and activate Wnt signaling. In addition to naturally-occurring allelic variants of the Wnt sequences that may exist in the population, it will be appreciated that, as is the case for virtually all proteins, a variety of changes can be introduced into the sequences without substantially altering the functional (biological) activity of the polypeptides. Such variants are included within the scope of the terms “Wnt”, “Wnt protein” and the like. Wnts are related to one another in sequence and strongly conserved in structure and function across multiple species. Thus a Wnt protein displaying activity in one species may be used in other species to activate the Wnt pathway in such species and may be expected to display similar activity. Wnt family members include Wnt1, Wnt2, Wnt2b (also called Wnt13), Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt9a, Wnt10b, Wnt11, Wnt16, Wnt10a, Wnt10b, Wnt11, Wnt14, Wnt15, or Wnt1. Sequences of Wnt genes and proteins are known in the art. One of skill in the art can readily find the Gene ID, accession numbers, and sequence information for Wnt family members and other genes and proteins of interest herein in publicly available databases. The Wnt protein may be isolated from naturally occurring sources (e.g., mammalian or insect cells that naturally produce the protein), produced in eukaryotic or prokaryotic cells using recombinant expression technology, or chemically synthesized. Soluble, biologically active Wnt proteins may be prepared in purified form using methods known in the art. See, e.g., U.S. Pat. Pub. No. 20040248803 and Willert, K., et al., Nature, 423: 448-52, 2003. In certain embodiments the soluble, biologically active Wnt protein is Wnt3a. In certain embodiments the Wnt protein is co- or post-translationally modified as occurs when the Wnt protein is produced in a host cell that naturally expresses the Wnt protein. In other embodiments the Wnt protein is not co- or post-translationally modified as in nature. In certain embodiments the soluble, biologically active Wnt protein is modified with a lipid moiety such as palmitoylate. The lipid moiety may be attached to a conserved cysteine. For example, in certain embodiments the Wnt protein is palmitoylated on a conserved cysteine as known in the art. In certain embodiments the Wnt protein is glycosylated as occurs when the Wnt protein is produced in a mammalian host cell that naturally expresses the Wnt protein. In other embodiments the Wnt protein is not glycosylated as found in nature. Recombinant mouse Wnt3a is commercially available (e.g., from Millipore cat. no. GF 145 or R& D Systems cat. no. 1324-WN-002).

Wnt3a is preferably produced in cell culture, like in a system using insect cells or using mammalian cells (Willert, K. et al, supra) or the system as described in U.S. Pat. No. 7,153,832, which herewith is incorporated by reference. From these the protein then can be isolated. Wnt3a can be present in a concentration of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375, about 400, about 450, about 500, about 550, about 600, about 650, about 700, about 750, about 800, about 850, about 900, about 950 or about 1000 ng/ml. Higher concentrations can be present as well.

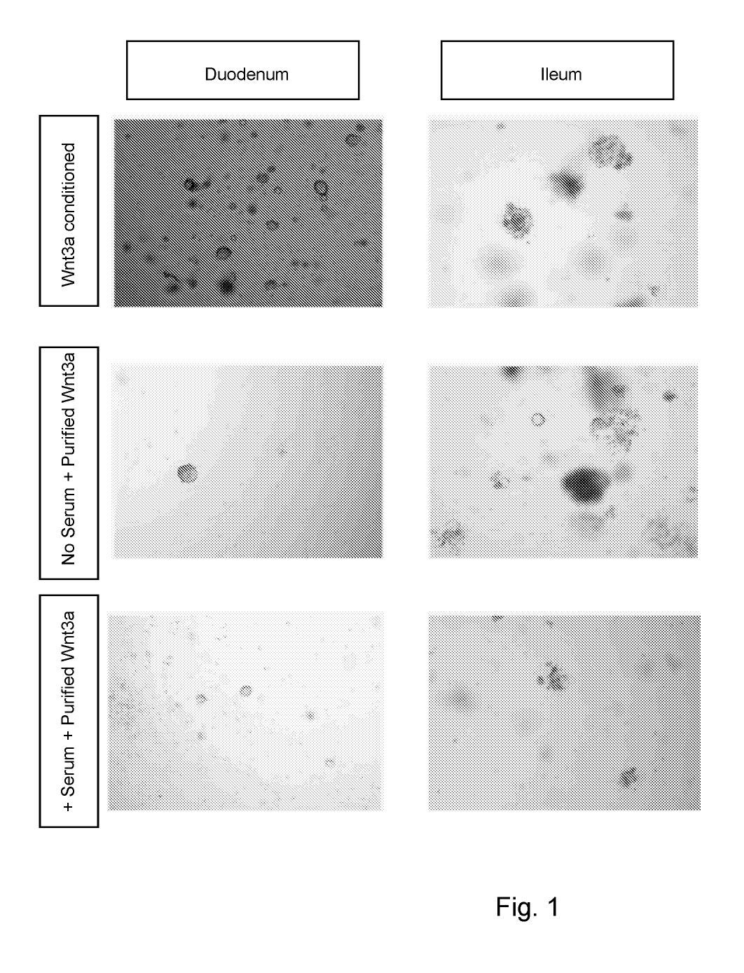

Wnt3a is a protein that is used very advantageously in the culturing of adult stem cells. However, when adding purified Wnt3a protein in human duodenum and ileum organoid cultures in serum-free conditions, these cultures were lost after 2 passages. When Wnt-conditioned medium was used, i.e. medium that contained serum, organoid cultures were successfully established and maintained (FIG. 1). Since Wnt3a-conditioned medium contains a high percentage of serum, it was further tested whether addition of serum would improve the establishment of organoids using purified Wnt3a. Indeed, addition of serum together with purified Wnt3a protein improved the derivation efficiency of both duodenum and ileum organoid cultures, although not to the level achieved with Wnt3a-conditioned medium (FIG. 1). These data suggest that serum promotes organoid culture in combination with Wnt3a. Recent data indicates that serum stabilizes Wnt3a protein activity (Fuerer et al., supra), and therefore it was further investigated whether reduced Wnt activity in serum-free conditions explained its failure to support organoid derivation.

Establishment and maintenance of adult stem cell cultures appeared possible by adding a fatty substance to (the medium comprising) the Wnt protein in a concentration of at least 0.1 mM. Such a fatty substance is preferably a phospholipid or a lipid with detergent activity, i.e. an amphipathic molecule, supplied in the form of a liposome or a micelle. The biological effect, i.e. stabilisation of the Wnt protein activity, is not only achieved if the liposome or micelle is associated with the Wnt protein and then added to the culture medium, but is also achieved when protein and fatty substance are added separately to the medium. With regard to micelles, it should be emphasized that care should be taken, when adding detergents in the form of micelles, that the concentration is such that the micelles will be maintained when introduced into the larger volume of the culture medium. This means that the micelles that are given should have a relatively low critical micelle concentration. The skilled person will know how to prepare micelles with such a low critical micelle concentration.

A wide range of lipids can be used for making the liposomes or micelles. Lipids and phospholipids that are normally used for liposome preparation may be used. Especially suitable components for forming liposomes are phosphatidylcholines, such as 1,2-Dimyristoyl-sn-Glycero-3-Phosphocholine (DMPC), 1,2-Dipalmitoyl-sn-Glycero-3-Phosphocholine (DPPC), 1-Myristoyl-2-Palmitoyl-sn-Glycero-3-Phosphocholine (MPPC), 1-Palmitoyl-2-Oleoyl-sn-Glycero-3-Phosphocholine (POPC) and combinations thereof. As is shown in Morrell et al. (PLos ONE 2008, 3:e2930) DMPC appears to be the optimal choice out of the above-mentioned lipids. Nevertheless, it is believed that other lipids, such as lysophophatidylcholines, phosphatidylinositols, phosphatidylethanolamines, phosphatidylserines, sterols, like cholesterol, or sphingolipids, like sphingomyelin, saccharolipids and PEGylated phospholipids and further variants of the above mentioned lipids—having variations in the length of the acyl chain, in the amount of saturation, in the nature of the polar headgroup and charge, etcetera—will alternatively be useful. With regards to lipid molecules that may be used in the form of micelles, any kind of surfactants or detergents may be used, such as soaps, linear alkylbenzenesulfonates, lignin sulfonates, fatty alcohols and alkylphenolic compounds. Examples for this category are SDS, octylthioglucosides and CTAB (cetrimonium bromide).

The liposomes, micelles or other lipid aggregates that may be used in the present invention may also be formed by combination of the above-mentioned lipid molecules. It is very common, for instance, that liposomes are made out of two or three different lipid molecules. Generally, the components involved will be a phosphatidylcholine compound as basis which is stabilized by a phosphatidylglycerol compound and a sterol, such as cholesterol. A nice overview of the different forms of liposomes and how to prepare them is given in Akbarzadeh, A. et al., Nanoscale Res. Lett. 2013, 8:102, doi:10.1186/1556-276X-8-102).

The production method, the size of the liposome or micelle, the concentration ratio between Wnt protein and liposome or micelle, and the presence or absence of further detergents, cryoprotectants and further excipients for stabilizing protein structure do not seem to be critical. Apparently these can be varied within large ranges. However, the Wnt proteins are most stable when the final lipid concentration in the tissue culture medium exceeds 0.1 mM. Therefore, the lipid concentration in the tissue culture medium maybe more than 0.1 mM, more than 0.2 mM, more than 0.3 mM, more than 0.4 mM, more than 0.5 mM, more than 0.6 mM, more than 0.7 mM, more than 0.8 mM, more than 0.9 mM or higher. Even values of more than 1.0 mM to more than 2 mm to more than 3 mM and up to 10 mM may be used without hampering the beneficial effect of the lipid on Wnt protein stability.

Natural or recombinant Wnt proteins or peptides or variations thereof that mimic the activity of Wnt proteins can be used. The Wnt proteins can be in association with other proteins, e.g. lipoproteins or glycoproteins, or with other molecules that support their activity or solubility.

Culture medium which has been supplied with the above-mentioned lipid compositions, be it associated with the Wnt protein or added separately from the Wnt protein, would further contain the normal ingredients to be found in stem cell culture medium, except for serum. Accordingly, the culture medium may further contain buffer compounds, detergents, bulking agents, nutrient compounds, growth factors and other chemical or biological compounds that may be needed for maintaining and/or proliferation of stem cells, especially adult stem cells. The culture may also contain extracellular matrix components such as collagens, laminins, fibronections, vitronectins and other macromolecules, or mixtures thereof, of biological or synthetic origin, e.g. matrigel, hyalyronic acid, a poly(D,L-lactide-co-glycolide) fiber matrix, a polyglactin fiber, a calcium alginate gel, hydrogels, silicone compounds, or other synthetic polymers.

As indicated above, stabilisation of the adult stem cells with liposomes or other lipid aggregates, now makes it possible to culture said stem cells in a medium without serum. This would enormously increase the availability of such stem cells for therapeutic purposes, especially in the field of stem cell therapy. Accordingly, the invention comprises a method for the culturing of adult stem cells in a culture medium according to the invention, i.e. a medium in which a Wnt protein and a lipid are comprised.

It has been shown in the past for several stem cells that they can be transplanted into a recipient. Such transplanted stem cells can permanently engraft and perform their normal functions. An existing clinical application is the transplantation of bone marrow,which contain contains hematopoietic stem cells, into leukemia patients. The transplanted stem cells generate healthy blood cells that replace the cancerous tissue end permanently cure the patient. Research into solid tissue stem cells has not made the same progress as haematopoietic stem cells because of the difficulty of reproducing the necessary and precise 3D arrangements and tight cell-cell and cell-extracellular matrix interactions that exist in solid organs. Yet, the ability of tissue stem cells to assimilate into the tissue cytoarchitecture under the control of the host microenvironment and developmental cues, makes them ideal for cell replacement therapy. Transplantation and engraftment for several other types of (adult) stem cells, have ben demonstrated in animal models and are currently being tested for human therapy.

The diseases and field in which such stem cell therapies can be used (and have been demonstrated to work in animal studies or in incidental human treatments) are numerous. The Wikipedia article on stem cell therapy nowadays lists a multitude of areas or organs from which adult stem cells have been derived and used for curing diseases affecting those organs, such as corneal stem cells for treatment of blindness, cochlear stem cells for treatment of deafness, neural stem cells for treatment of Parkinson's disease or Alzheimer's disease, adipose-derived stem cells for treatment of myocardial infarction, mesenchymal stem cells for treatment of orthopedic defects, and so on. Of course all of these therapies would also be suitable in veterinary applications.

Accordingly, the invention comprises a method for stem cell therapy with adult stem cells, where the adult stem cells have been cultured in a culture medium according to the invention.

One of the major drawbacks in the field of stem cell therapies is formed by the immune reactions that can be caused by the application of the stem cells. Therefore, increasingly, it is tried to evolve therapies that make use of the patient's own stem cells, also called autologous stem cell transplantation. It will be clear that such therapies using the patient's own stem cells will also be enhanced by the current invention. The isolation and culturing of stem cells from the biopsies taken from the patient will be much improved and easier with the culture medium of the invention.

Accordingly, the invention comprises a method for autologous stem cell transplantation, which comprises the steps of isolating stem cells form a subject (wherein the subject may be an animal or a human), in vitro culturing said stem cells in a culture medium according to the invention, and reintroducing said cultured stem cells back into the same subject.

Although less desirable because of the potential immunogenicity reaction, all these therapies may also be possible using allogeneic stem cells. Also, tissue-specific stem cells may be derived from embryonic stem cells or induced pluripotent stem cells, which are capable of generating all tissues of the body including adult stem cells. Induced pluripotent stem cells can be obtained by expressing reprogramming factors, frequently Oct4, Sox2 and Klf4 but others are possible as well, in cells from a human or animal donor. This way, cells from a patient or a donor can be reprogrammed into pluripotent cells and these can then be differentiated into tissue specific stem cells.

Wnt liposomes may not only be helpful in establishing and maintaining adult stem cell cultures but also in directing differentiation of embryonic stem cells, induced pluripotent stem cells, or other stem cells into mature cell types or other stem cell types.

In autologous stem cell transplantation it is also possible to treat the stem cells that have been isolated from the body to modify them. In many cases such a treatment will be a specific genetic modification. Such a stem cell dependent gene therapy has been described in the literature (e.g. Watts, K. et al., 2011, Cytotherapy 13:1164-1171 and Kohn, D. et al., 2013, Biol. Blood Marrow, Transplant, 19:S64-S69 for hematopoietic stem cells and San, S. et al., 2010, Hum. Gene Ther. 21:1327-1334 for endothelial precursor cells). AS has been nicely worded in the article of Kohn et al., efforts to date have focused on stable addition of a replacement gene (cDNA, globin mini-locus, or genomic segment) or in situ modification of the endogenous gene. The diseases that were targeted were primary immune deficiencies, hemoglobinopathies and lysosomal storage disorders. It will be clear that the method of culturing stem cells according to the present invention also enables a better autologous stem cell therapy and provides opportunities for isolating and culturing the stem cells for genetic modification.

The culture method of the present invention is especially advantageous in this respect, since it allows to easily isolate clones with the correct genetic modification and to verify the absence of genetic abnormalities. These steps may additionally be performed according to a method of autologous stem cell therapy of the invention.

Irrespective of the culturing method, it is postulated that genetically modified adult stem cells would not only be useful in therapies for disease which relate to the organ from which the stem cell has been derived, but such stem cells would also be useful for producing compounds that are effective against diseases in other organs. Accordingly, part of the present invention is a method for autologous adult stem cell therapy in which the adult stem cell is isolated from an organ of a subject (may be human or animal), subsequently genetically modified and reintroduced into the subject, whereby the genetic modification causes the stem cell to produce a therapeutic compound, wherein said compound is effective against a disease of another organ than the organ from which the stem cell is derived. It should be understood that the term ‘organ’ as used herein is used for the indication of a tissue or collection of tissues that serve a common function and which may be joined in a structural unit. The term ‘structural unit’ should be interpreted more broadly than structure, since some organs, such as skin or blood do not form a single, confined structure. The organs are joined in a broader functional unit, organ system, which in the case of blood is formed by the cardiovascular system, which also comprises the heart and blood vessels. In a more limited sense the invention comprises a method for autologous adult stem cell therapy in which the adult stem cell is isolated from an organ of a subject (may be human or animal), subsequently genetically modified and reintroduced into the subject, whereby the genetic modification causes the stem cell to produce a therapeutic compound, wherein said compound is effective against a disease of another organ system than the system from which the organ from which the stem cell is derived belongs.

The therapeutic factors that are produced by the genetically modified stem cells may be any therapeutically effective compound and can be formed by proteins, but may also be formed by secondary metabolites. Based on their pharmacological activity, they can be divided into five groups: (a) replacing a protein that is deficient or abnormal; (b) augmenting an existing pathway; (c) providing a novel function or activity; (d) interfering with a molecule or organism; and (e) a targeting moiety for the previous groups of effector proteins. Therapeutic proteins can also be grouped based on their molecular types that include antibody-based drugs, Fc fusion proteins, anticoagulants, blood factors, bone morphogenetic proteins, engineered protein scaffolds, enzymes, growth factors, hormones, interferons, interleukins, and thrombolytics. They can also be classified based on their molecular mechanism of activity as (a) binding non-covalently to target, e.g., mAbs; (b) affecting covalent bonds, e.g., enzymes; and (c) exerting activity without specific interactions, e.g., serum albumin. Stem cells can be genetically modified to produce such a therapeutic protein. When re-introduced into the body, the stem cell will develop into a cell in an organ of the recipient and start producing the protein. The protein then will enter the lymph stream and/or the blood and will reach blood levels that would also be reached by ‘normal’ administration of the biological compound, such as oral or parenteral administration. Supply of the therapeutic protein with the help of stem cells may lead to a permanent cure of a disease since stem cells may remain in the body for the rest of the lifetime of the recipient. A specific example of a stem cell therapy with a protein that may be produced by a genetically altered adult human stem cell is the use of intestinal stem cells that are genetically modified to be able to produce an enzyme called acid alpha-glucosidase (GAA) which is used as a therapy for Pompe's disease (glycogen storage disease type II). In this case thus adult stem cells that were derived from the intestine are genetically altered and used to cure a disease which is not at all related to the organ (intestine) from which the cells were derived. Pompe's disease normally mainly affect the unrelated skeletal muscle and heart muscle cells. A similar approach can be taken for other autosomal or other inherited diseases where therapy may be formed by therapy with the normal gene product, such as diabetes (administration of insulin), phenylketonuria (administration of the enzyme phenylalanine lyase, PAL) and growth hormone deficiencies, where therapy is formed by administration of growth hormone. It may be obvious that therapies may both address problems in the organ into which the cultured stem cells are placed and in unrelated organs.

The same effect as described herein for proteins may also be obtained by the genetic transformation of stem cells where the genetic transformation results in the production and excretion of secondary metabolites. Compounds that could be produced in such a way are hormones, like oestrogens, which would be useful in therapy of breast or ovarian cancer and for hormonal regulation during and after menopause and in sex reassignment therapy. Alternatively, production of a hormone antagonist could lead to a long-lasting sterilisation or even chemical castration. Also production of cortisol and aldosterone can be used, e.g. to treat Addison's disease.

The invention is further illustrated by the following examples. The examples are not to be interpreted as limiting the scope of the invention in any way.

### EXAMPLES

### Materials and Methods

### Derivation of Intestinal Stem Cell Cultures

Organoid cultures were established from fresh human duodenum and ileum tissue samples as described (Sato, T. et al., 2011, Gastroenterol. 141:1762-1772). In short, the intestinal tissues were washed and stripped of the underlying muscle layers with surgical scissors. The tissue was chopped into approximately 5-mm pieces and further washed with cold PBS. Next, the tissue fragments were incubated in 2 mmol/L EDTA cold chelation buffer (distilled water with 5.6 mmol/L Na2HPO4, 8.0 mmol/L KH2PO4, 96.2 mmol/L NaCl, 1.6 mmol/L KCl, 43.4 mmol/L sucrose, 54.9 mmol/L d-sorbitol, 0.5 mmol/L dl-dithiothreitol) for 30 minutes on ice. After removal of the EDTA buffer, tissue fragments were vigorously resuspended in cold chelation buffer using a 10-mL pipette to isolate intestinal crypts. The tissue fragments were allowed to settle down under normal gravity for 1 minute, and the supernatant was removed for inspection by inverted microscopy. The resuspension/sedimentation procedure was typically 6-8 times, and the supernatants not containing crypts were discarded. The supernatants containing crypts were collected in 50-mL Falcon tubes coated with bovine serum albumin. Isolated crypts were pelleted, washed with cold chelation buffer, and centrifuged at 150-200 g for 3 minutes to separate crypts from single cells. They were then embedded in Matrigel on ice (growth factor reduced, phenol red free; BD Biosciences) and seeded in 48-well plates (500 crypts/fragments or 1000 single cells per 25 μL of Matrigel per well). The Matrigel was polymerized for 10 minutes at 37° C., and then flooded with 250 μL/well basal culture medium (advanced Dulbecco's modified Eagle medium/F12 supplemented with penicillin/streptomycin, 10 mmol/L HEPES, Glutamax, 1×N2, 1×B27 [all from Invitrogen], and 1 mmol/L N-acetylcysteine [Sigma]) containing 50 ng/ml murine EGF, 100 ng/ml murine noggin, 1 μg/ml human R-spondin-1, 1 mM gastrin, 10 mM nicotinamide, 500 nM A83-01, 10 μM SB202190. The medium was further supplemented with either 50% Wnt3a conditioned medium or 250 ng/ml purified Wnt3a protein or 10% fetal calf serum together with 250 ng/ml purified Wnt3a protein, as indicated.

The entire medium was changed every 2 days and organoids were passaged 1:5 every week. For passage, the culture medium was replaced with fresh basal culture medium. Organoids and Matrigel were mechanically disrupted using a P1000 pipette and transferred into a 15-ml falcon tube. Further mechanical dissociation was achieved using a firepolished pasteur pipette. Dissociated organoids were washed with 10 ml of basal culture medium and centrifuged at 200 g for 2 min. The supernatant was discarded, the pellet resuspended with Matrigel and culture medium was added as described above.

### Production of Wnt3a Conditioned Medium

To produce Wnt3a conditioned medium, L-Wnt3a cells (ATCC CRL-2647) were grown to confluency, trypsinized, and replated at a 6-fold larger surface in DMEM medium supplemented with 10% fetal calf serum. After 1 week the medium was collected, centrifuged at 15000 rpm for 5 min to remove floating cells, filtered through a 0.22 micrometer filter, and stored at 4° C. until use.

### Purification of Wnt3a Protein

Recombinant mouse Wnt3a protein was produced in Drosophila S2 cells grown in suspension culture, and purified by Blue Sepharose affinity and gel filtration chromatography as described (Willert, Brown et al. 2003).

### Activity Assays for Purified and Liposomal Wnt3a Reagents

Mouse LSL cells, expressing luciferase in response to activation of the Wnt pathway (Mikels, A. and Nusse, R., 2006, PLos Biol. 4:e115), were cultured at 37° C. and 5% CO₂ in DMEM, 10% FBS, and 1% Penicillin/Streptomycin. For the activity assays, 25,000 LSL cells/well were plated in 96-well plates and grown for 24 hours. The cells were then treated with the Wnt reagents which were separately incubated in DMEM, 10% fetal calf serum, 1% Penicillin/Streptomycin or in DMEM, 1% Penicillin/Streptomycin medium at 37° C. in 96-well plates for various periods of time. After an additional overnight incubation with the indicated reagents, luciferase activity was measured using Luciferase assay reagent (Promega) according to the manufacturer's instructions. Activity is plotted relative to control LSL cells as the average of 3 samples.

### Embryonic Stem Cell Assays

ES cells were maintained in N2B27 medium on plates coated first with gelatin, followed by a coating with fetal calf serum. N2B27 medium (Ying, Q. et al., 2003, Nat. Biotechnol. 21:183-186) consisted of 1 volume DMEM/F12 combined with 1 volume Neurobasal medium, supplemented with 0.5% N2 Supplement, 1% B27 Supplement, 0.033% bovine serum albumin 7.5% solution, 50 μM beta-mercaptoethanol, 2 mM Glutamax, 100 Units/ml penicillin and 100 μg/ml streptomycin (all from Invitrogen). Cells were passaged as a single cell suspension using 0.25% Trypsin-EDTA. After passaging, trypsin was quenched using soybean trypsin inhibitor (Sigma).

To quantify self-renewal over multiple passages, single cells were plated at a density of 100 cells/cm² in gelatine- and serum-coated 6-wells plates and in gelatine- and serum-coated 24-wells plates in triplicate. Every 3 days, the 6-wells plates were trypsinized to single cells, and passaged to a new set of plates at a dilution that would lead to a density not higher than but as close as possible to 100 cells/cm². At the same time, the 24-wells plates were stained for alkaline phosphatase using the SCR004 kit (Millipore). Stained plates were rinsed with water, dried, and the number of positive colonies manually counted. The cumulative number of colonies was determined by multiplying the colony counts by the dilution factor used for passaging. Results are plotted as the mean of 3 wells+/−s.e.m.

### Preparation of Wnt3a Liposomes

For preparation of liposomes, different kinds of phospholipids were tested. In all cases, the final concentration of phospholipids (not including cholesterol) was kept the same. Dimyristoyl-sn-Glycero-3-Phosphocholine (DMPC), DMPG (1,2-dimyristoyl-sn-glycero-3-phospho-(1′-rac-glycerol) were obtained from Lipoid AG (Ludwigshaven, Germany). Cholesterol was obtained from Sigma Aldrich Co. LLC (st. Louis, Mo. USA).

For the preparation of Wnt3a liposomes, first lipids were mixed at certain molar ratios indicated in brackets: DMPC/DMPG (10:1), DMPC/DMPG/Cholesterol (10:1:1), DMPC/DMPG/Cholesterol (10:1:4), DMPC/DMPG/Cholesterol (10:1:10). The lipid mixtures were dissolved in chloroform/methanol in a ratio of 9/1 (v/v). The organic phase was then gradually evaporated under vacuum on a rotavapor until a film layer formed. The residual organic solvent was removed by nitrogen gas flushing. The lipid film was then suspended in HBS at a concentration of 88 mM phospholipid. The lipid suspension was extruded 10 times through two stacked polycarbonate filters with a pore size of 200 nm and 100 nm, respectively, under nitrogen pressure using a Lipex high-pressure extruder. Final phospholipid concentration was determined by phosphate assay. The size and dispersity of the liposomes was determined by dynamic light scattering.

For association with liposomes, 50-80 ug/ml purified Wnt3a protein in 1% CHAPS in PBS was mixed with liposomes and PBS to a total concentration of 7-10 ug/ml Wnt3a and 18.5 mM phospholipid. The mixture was then incubated for one hour on the roller coaster at 4° C. CHAPS was removed from the Wnt liposomes by dialysis in PBS three times for 1 hour each, using dialysis membrane with molecular weight cut-off of 10 kD at 4° C.

Determination of CHAPS Content

CHAPS concentrations were determined on HPLC (Aliance Waters 2695, Waters, USA), using reversed phase chromatography and UV detection (Dual λ Absorbance detector, Waters, USA) at 210 nm. The column was LiChrospher 100, RP-18 (5 μM). As a mobile phase, 4% Acetonitril, 95.9% water, and 0.1% perchloric acid was used, and the flow rate was 1.0 ml/min. The calibration curve ranged from 50 to 1000 μg/ml.

### Example 1

### Wnt3a Protein Rapidly Loses Activity in Serum-Free Media

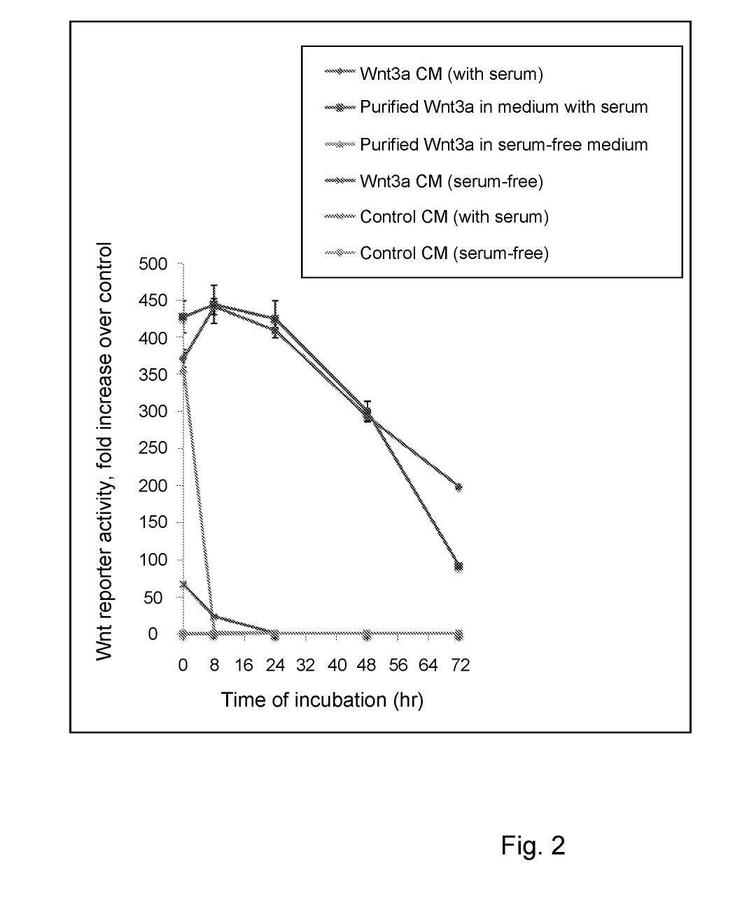

To assess its stability, Wnt3a protein was incubated for various times in cell culture medium at 37° C., and the remaining activity was assayed using the LSL reporter assay (Mikels A. and Nusse R., 2006, PLos Biol. 4:e115). LSL cells contain a luciferase reporter driven by a Wnt responsive promoter, allowing a quantitative readout of Wnt activity. These assays demonstrated that purified Wnt3a loses its activity within a few hours in serum free medium, while in the presence of serum this period is extended to more than a day (FIG. 2). Moreover, Wnt3a-conditioned medium retains its activity for several days (FIG. 2). Thus, the rapid loss of Wnt3a activity in serum-free conditions may explain why these conditions fail to support organoid cultures.

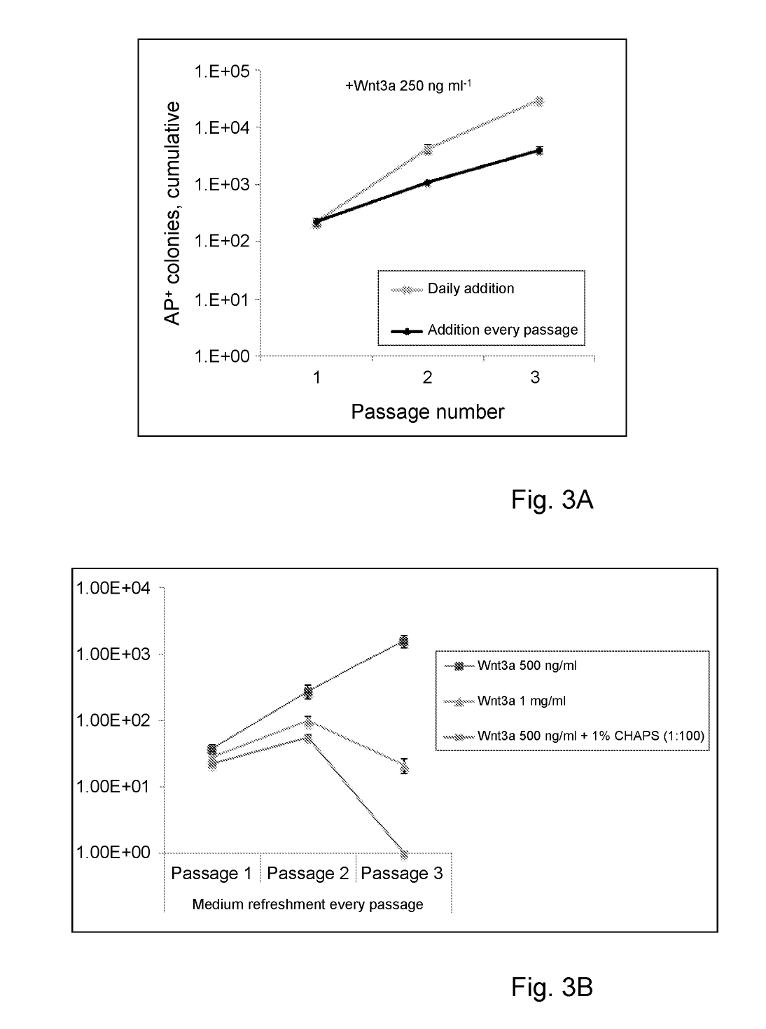

To quantify the effect of Wnt half-life on stem cell self-renewal we made use of a serum-free mouse embryonic stem cell (ESC) self-renewal assay, which is extremely sensitive to the level of Wnt activity in the culture (ten Berge, D. et al., 2011, Nat. Cell Biol. 13:1070-1075). We found that ESC self-renewal significantly declined when Wnt3a was added every 3 days instead of daily (FIG. 3a), indicating that the short half-life of Wnt3a protein limits its ability to support ESC self-renewal. When we tried to overcome this limitation by increasing the amount of Wnt3a protein in the culture, we observed that above a certain threshold, larger concentrations of Wnt3a repressed self-renewal (FIG. 3b). This however appeared to be due to the toxic effect of the detergent CHAPS (FIG. 3b), which is present at a high concentration in purified Wnt3a protein and required to maintain its activity (Willert, K. et al., 2003, Nature 423:448-452). Thus, Wnt3a activity not only declines rapidly in serum-free cell culture, but the presence of CHAPS in Wnt protein preparations places a strict ceiling on the amount of Wnt3a protein that can be added to cell cultures and prevents frequent addition of fresh Wnt3a protein to maintain activity.

### Example 2

### Association with Lipid Vesicles Stabilizes Wnt3a Protein Activity

To prepare Wnt3a liposomes, a suspension of different lipids was first extruded to obtain uniformly sized liposomes. These were then mixed with Wnt3a protein to allow association of the protein with the liposomes. We showed previously that presence of the phospholipid DMPC in the liposomes is beneficial to maintain Wnt3a activity (Morell, N. et al., 2008, PLos One 3:e2930). Due to the absence of charge, pure DMPC liposomes tend to aggregate which can be prevented by adding a charged phospholipid, such as DMPG. The presence of cholesterol can further enhance the physical stability of liposomes. After preparation, the liposomes were analyzed for their size, polydispersity index, Wnt3a content, CHAPS content, and Wnt3a activity.

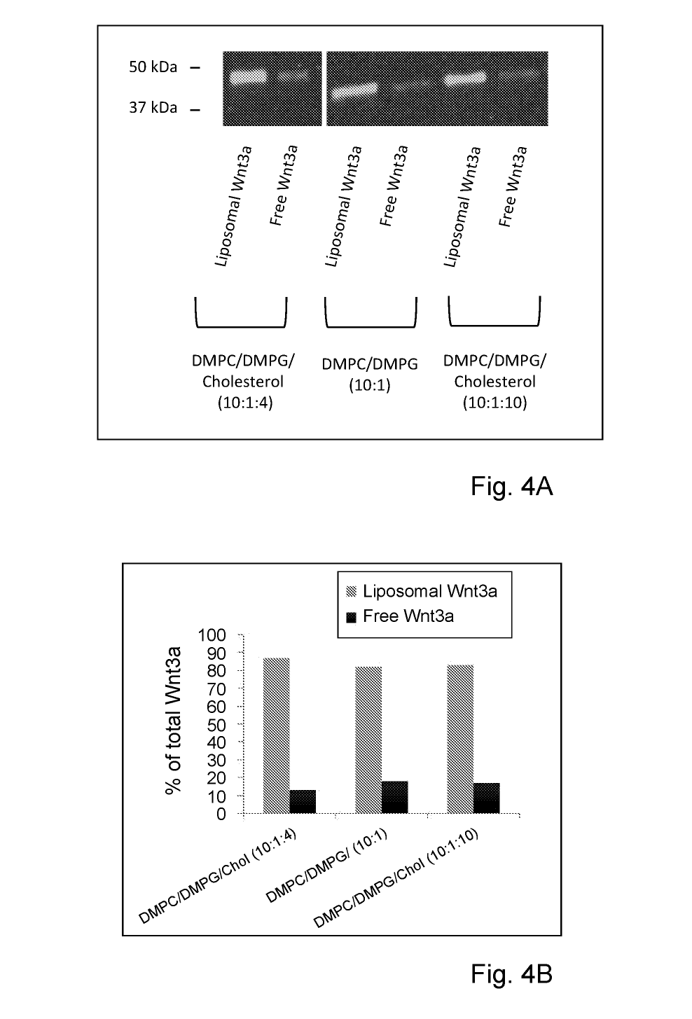

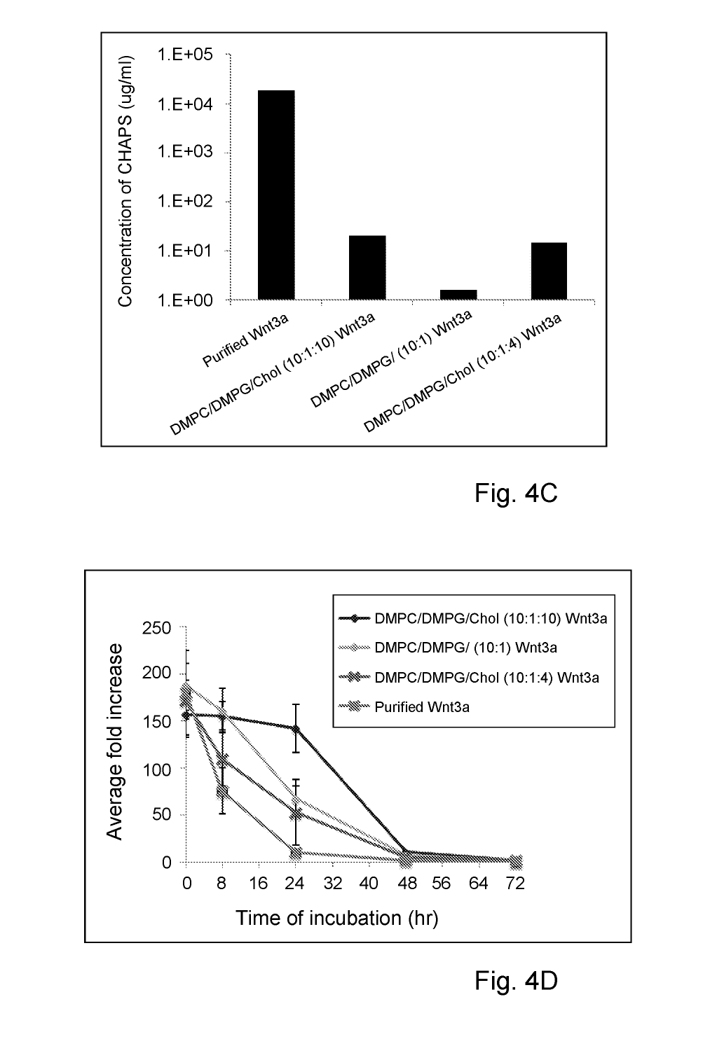

Size and polydispersity index measurements showed that association with Wnt3a did not affect these physical properties of the liposomes. To determine the incorporation of Wnt3a protein, liposomes were spun down by ultracentrifugation and the Wnt3a content of liposomes and supernatant quantified by western blotting (FIG. 4a). Between 82% to 87% of total Wnt3a protein was found to be associated with the liposomes (FIG. 4a,b), in agreement with earlier measurements (Morell et al., supra). Subsequent dialysis of the Wnt3a liposomes successfully lowered CHAPS concentration to negligible levels (FIG. 4c). Moreover, activity measurements using the LSL assay showed that association with liposomes considerably prolonged the activity of Wnt3a in serum-free medium, in particular when using liposomes composed of a mixture of DMPC:DMPG:Cholesterol in a 10:1:10 ratio (FIG. 4d). These data indicate that the incorporation of Wnt3a protein into lipid vesicles stabilizes its activity, even in the absence of CHAPS, potentially enabling us to provide longer lasting Wnt stimuli to cells. In addition, by removing the need for the toxic detergent CHAPS it becomes possible to add larger amounts of Wnt3a to the cultures.

Liposome-Stabilized Wnt Ligands Provide Superior Support for Serum-Free Stem Cell Cultures

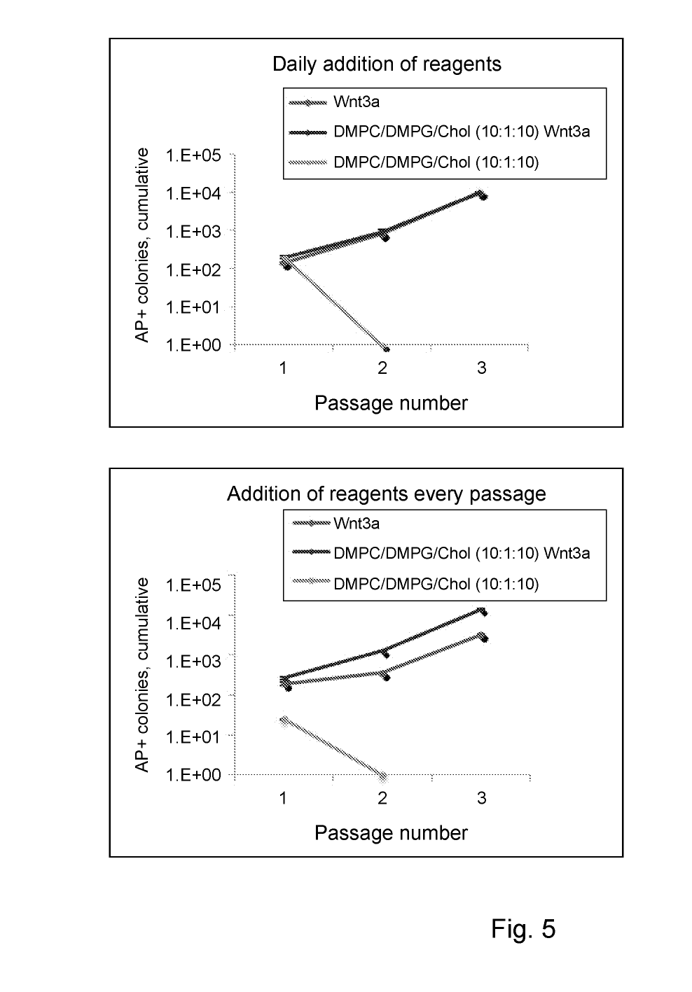

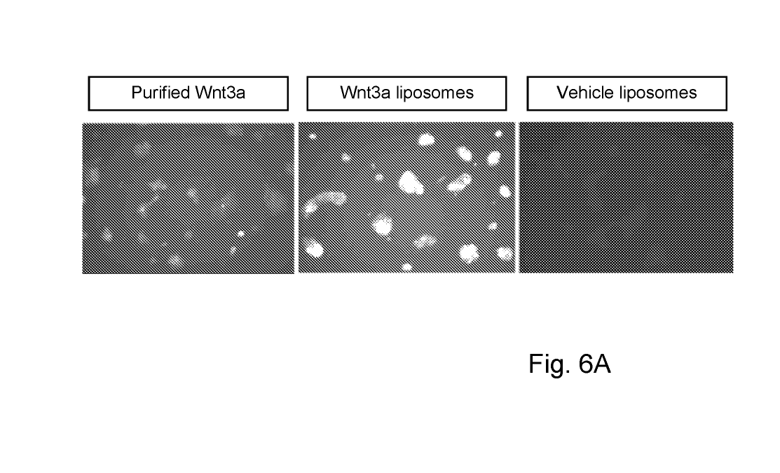

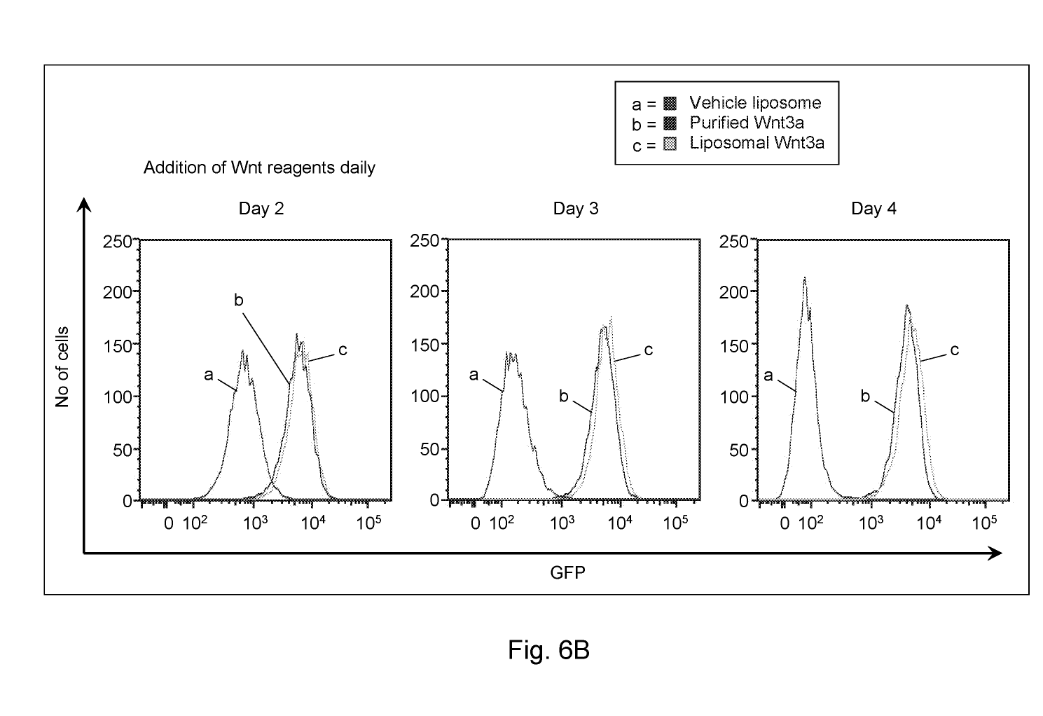

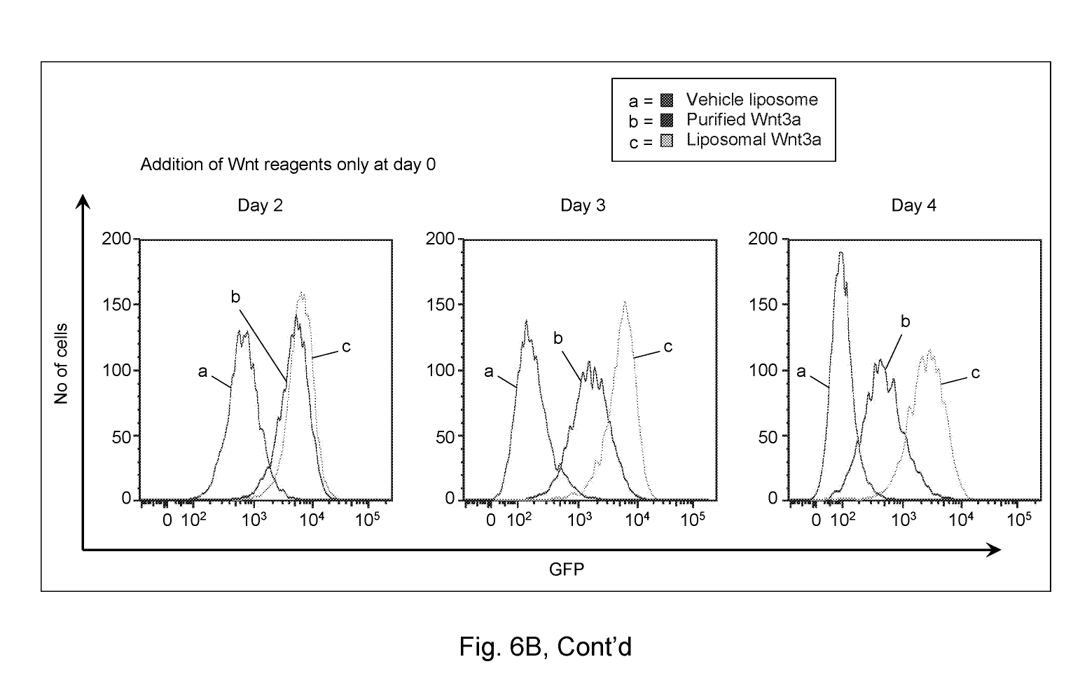

We initially assessed the functional performance of Wnt3a liposomes in ESC self-renewal assays. This showed that a single addition of Wnt3a liposomes to the cells following their passaging every three days promoted a 3-fold higher expansion of undifferentiated cells than purified Wnt3a (FIG. 5). Moreover, by using R1-7xTcf-eGFP cells, which carry a Wnt-responsive GFP reporter (ten Berge, D. et al., 2008, Cell Stem Cell 3:508-518), we found that Wnt3a-liposomes maintained strong reporter activity even 4 days after addition, while reporter activity started to decline steadily after the first day of adding purified Wnt3a (FIG. 6a,b). These data show that the prolonged activity of the novel Wnt3a liposomes translates into a considerably increased capacity to maintain target gene activation and to support stem cell expansion.

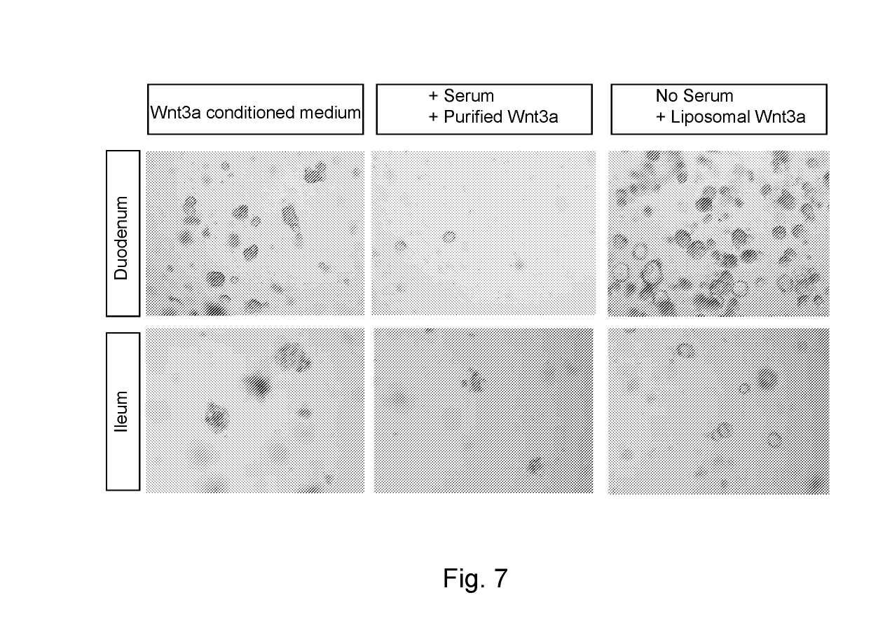

Finally, we tested the ability of Wnt3a liposomes to support the derivation and maintenance of human duodenum and ileum organoid cultures. In contrast to purified Wnt3a, the Wnt3a liposomes supported efficient establishment and maintenance of organoid cultures from both tissues in serum-free conditions (FIG. 7). Moreover, the Wnt3a liposomes also strongly enhanced organoid derivation relative to Wnt3a-conditioned medium or purified Wnt3a in combination with serum, and showed considerably reduced evidence of differentiation (FIG. 7). This indicates that factors in serum and in Wnt3a-conditioned medium promote differentiation of the organoid stem cells. These data show that Wnt3a liposomes not only enable the establishment of organoid cultures in defined conditions, but also increase the efficiency of derivation and culture by eliminating differentiation-inducing factors from the culture system.

Association with Lipid Vesicles Stabilizes Wnt3a Protein Activity

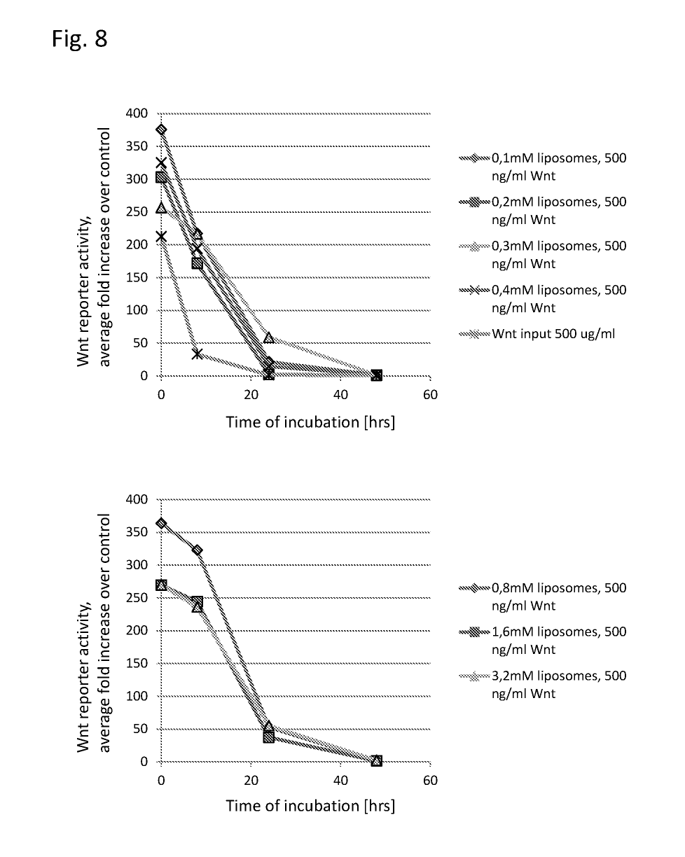

We further tested whether it was essential to complex the Wnt3a protein with the liposomes before adding it to the medium. We found that the liposomes also stabilized Wnt3a protein activity when added separately to the cell culture medium (FIG. 8). In addition, we found that the liposomes were effective in stabilizing Wnt3a protein when present in a concentration range varying from 0.1 mM to 3.2 mM total phospholipid (FIG. 8), and most likely also in concentrations outside this range.

1. A method for in vitro culturing of stem cells, wherein the cells are held in a serum-free culture medium comprising a Wnt protein and a lipid and wherein said lipid is available in a concentration of at least 0.1 mM.

2. The method according to claim 1, wherein the lipid is in the form of a liposome or of a micelle.

3. The method according to claim 1, wherein the lipid and the Wnt protein are associated in a complex.

4. The method according to claim 3, wherein the liposome is composed of dimyristoyl-sn-Glycero-3-Phosphocholine (DMPC), 1,2-dimyristoyl-sn-glycero-3-phospho-(1′-rac-glycerol)) (DMPG) and cholesterol, preferably in a DMPC:DMPG:cholesterol ration of 10:1:10.

5. The method according to claim 1 wherein the Wnt protein is selected from the group of human Wnt protein, preferably wherein the protein is Wnt3a.

6. The method according to claim 1, wherein the stem cells are adult stem cells, preferably intestinal stem cells, more preferably stem cells obtained from duodenum and/or ileum.

7. The method according to claim 1, wherein the stem cell culture is an organoid culture.

8. A serum-free culture medium for the culture of stem cells comprising a Wnt protein and a lipid, wherein said lipid is available in a concentration of at least 0.1 mM, preferably wherein said lipid is in the form of a liposome or micelle.

9. A method for stem cell therapy, comprising the steps of:
a. isolation of stem cells from a subject;
b. optionally treating said stem cells, wherein the treatment may be chosen from differentiating, dedifferentiating, redifferentiation, reprogramming, introducing of a mutation, genetic modification;
c. culturing said stem cells in a serum-free culture medium as claimed in claim 8;
d. introducing said cultured stem cells in a subject in need thereof.

10. A method for autologous adult stem cell therapy comprising the steps of:
a. isolation of adult stem cells from a subject;
b. culturing said adult stem cells in a serum-free culture medium as claimed in claim 8;
c. re-introduction of said cultured adult stem cells in said subject.

11. A method for autologous adult stem cell therapy comprising the steps of:
a. isolation of adult stem cells from a subject;
b. genetic modification of said adult stem cells;
c. culturing said genetically modified adult stem cells in a serum-free culture medium as claimed in claim 8;
d. re-introduction of said cultured adult stem cells in said subject.

12. A method for adult stem cell therapy comprising the steps of:
a. isolation of adult stem cells from an organ of a subject;
b. genetic modification of said adult stem cells;
c. culturing said genetically modified adult stem cells in a serum-free culture medium as claimed in claim 8;
d. re-introduction of said cultured adult stem cells in said subject,
wherein said adult stem cells are genetically modified in such a way that they are producing a therapeutic compound for treatment of a disease wherein said disease is not or only partly related to the organ or the organ system from which the adult stem cells are derived in step a.

13. A method for adult stem cell therapy comprising the steps of:
a. isolation of adult stem cells from an organ of a subject;
b. genetic modification of said adult stem cells;
c. re-introduction of said cultured adult stem cells in said subject,
wherein said adult stem cells are genetically modified in such a way that they are producing a therapeutic compound for treatment of a disease wherein said disease is not or only partly related to the organ or the organ system from which the adult stem cells are derived in step a.

14. A serum-free medium comprising a Wnt protein and a lipid, wherein said lipid is available in a concentration of at least 0.1 mM, preferably wherein said lipid is in the form of a liposome or micelle, for adult stem cell therapy, wherein the adult stem cell therapy comprises:
a. isolation of adult stem cells from a subject;
b. culturing said adult stem cells in said serum-free culture medium;
c. re-introduction of said cultured adult stem cells in said subject.

15. A serum-free medium comprising a Wnt protein and a lipid, wherein said lipid is available in a concentration of at least 0.1 mM, preferably wherein said lipid is in the form of a liposome or micelle, for adult stem cell therapy, wherein the adult stem cell therapy comprises:
a. isolation of adult stem cells from a subject;
b. genetic modification of said adult stem cells;
c. culturing said adult stem cells in said serum-free culture medium;
d. re-introduction of said cultured adult stem cells in said subject.
